(19) United States
(12) Patent Application Publication
(10) Pub. No.: US 2006/0088489 A1
(43) Pub. Date: Apr. 27, 2006

(54) Sustained release of active molecules from polymers topically applied to skin or hair

(76) Inventors: Paolo Giacomoni, Commack, NY (US); Richard Gross, Plainview, NY (US); Stephen Laczynski, Pearl River, NY (US)

Correspondence Address: THE ESTEE LAUDER COS, INC, 125 PINELAWN ROAD, MELVILLE, NY, 11747, US

(21) Appl. No.: 11/259,458

(22) Filed: Oct. 26, 2005

Related U.S. Application Data
(60) Provisional application No. 60/622,583, filed on Oct. 27, 2004.

(51) Int. Cl. A61K 8/88; A61K 8/85

(52) U.S. Cl. 424/70.11; 424/70.17; 525/418

(57) ABSTRACT

The invention relates to a polymer for topical delivery of biologically active ingredients, the polymer comprising at least one moiety: U-B-A in which U represents a physiologically acceptable unit of an oligomer or polymer, A represents a biologically active component, and B represents one or more bond(s) linking A to U, which bond is capable of being disrupted by a biological, physical or chemical process occurring in or on skin.

This application claims priority from of U.S. 60/622,583, filed Oct. 27, 2004.

### FIELD OF THE INVENTION

The invention relates to topical compositions. More specifically, the invention relates to polymeric compositions useful in delivering biologically active materials to the skin and hair.

### BACKGROUND OF THE INVENTION

Topical application of enzymes, drugs, moisturizers, fragrances, and of other cosmetic or pharmacological molecules has been practiced for centuries in the course of human history. Topical Alpha-chemotrypsin is used to treat hematomas (1), topical salicylic acid at high concentration is used to remove callous bodies (2), whereas at low concentration it helps the natural process of desquamation to yield smooth skin surface (3) and topical vitamin E can be used to reduce the unwanted effects of solar radiation (4). Cosmetics and pharmaceuticals provide countless examples of beneficial effects obtained by topical administration of large variety of ingredients.

Transdermal delivery has recently gained popularity as a route of administration of both cosmetic and pharmaceutical actives, as an alternative to perfusion or systemic. Transdermal delivery has a number of advantages, which include less trauma to the patient in delivery, as well as enabling the use of drugs which, although efficient in treating specific diseases, are toxic for or disabled by the digestive system or which are not appropriate for the perfusion route. This method of delivering active material across the skin does have certain drawbacks and hurdles to overcome in its own right.

One of the major difficulties with transdermal delivery is that, to achieve effective delivery of the active, it often must permeate through the stratum corneum, the epidermis and the basal membranes of the skin. The success in achieving this is dependent upon the lipophilic/hydrophilic character of the material to be delivered (5). One method of circumventing this difficulty is the incorporation and delivery of the active in liposomes, and with liposomes, some success, at least for epidermal delivery, has been achieved (6).

Another major problem in achieving successful transdermal delivery is that a large amount of a drug needed to achieve the therapeutic result must be administered all at once, i.e., all at the moment of application. Depending upon the chemical identity of the active, the effective quantity can cause any number of undesirable effects, such as irritation, inflammation, local toxicity, or apoptosis. This effect is not limited to pharmaceuticals: similarly, suboptimal effects of cosmetic ingredients can also occur when they are applied to the skin or hair in a non-controlled manner. For example, an excess of moisturizer might not provide the desired feeling to dry skin, and an large quantity fragrance might be considered overwhelming or allergy-inducing to some particularly sensitive users.

Thus, there remains a continued need for development of systems for achieving sustained release of topically applied pharmacologic or cosmetic ingredients, with the desired result, among others, of prolonging the duration of the desired effects while avoiding adverse effects and/or expense of the application of large amounts of the free ingredient. The present invention provides a mechanism for achieving that goal.

### SUMMARY OF THE INVENTION

The present invention relates to a polymer for topical delivery of biologically active ingredients, the polymer comprising at least one moiety:
U-B-A
   in which U represents a physiologically acceptable unit of an oligomer or a polymer, B represents a bond capable of being disrupted by a biological, physical or chemical process occurring in or on skin, and A represents a biologically active component. As a result of disruption of the bond on the skin, the active ligand is released on the skin in a controlled fashion, rather than all being available simultaneously, and may thus result in a more benign and efficacious delivery of the material than would otherwise be achievable. The invention further provides topical compositions comprising the polymer of the invention, as well as a method of delivering a biologically active material to the skin, comprising applying to the skin a polymer of the invention containing the active material as a component.

### DESCRIPTION OF THE FIGURES

FIG. 1 is a diagram of the lipase-catalyzed synthesis of oligo ε-caprolactone) with geraniol esterified at the carboxyl termini of chains.

FIG. 2 is a diagram of the lipase-catalyzed condensation polymerization of sebasic acid, 1,8-octanediol, and anisyl to form poly(1,8-octanylsebacate) with anisyl esters at carboxyl termini of chains.

FIG. 3 is a diagram of the lipase-catalyzed condensation polymerization of adipic acid, sorbitol and anisyl alcohol to form the corresponding polyester with with anisyl esters at carboxyl termini of chains.

FIG. 4 is a diagram of the lipase-catalyzed synthesis of oligo(ε-caprolactone) with the 2-(4-aminophenyl)ethyl alcohol Schiff base derivative of floralozone at the carboxyl termini of chains

FIG. 5 is a diagram of the synthesis of floralozone glycerol acetal derivative and its conjugation by ester bonds to carboxyl chain ends during lipase-catalyzed synthesis of oligo(ε-caprolactone).

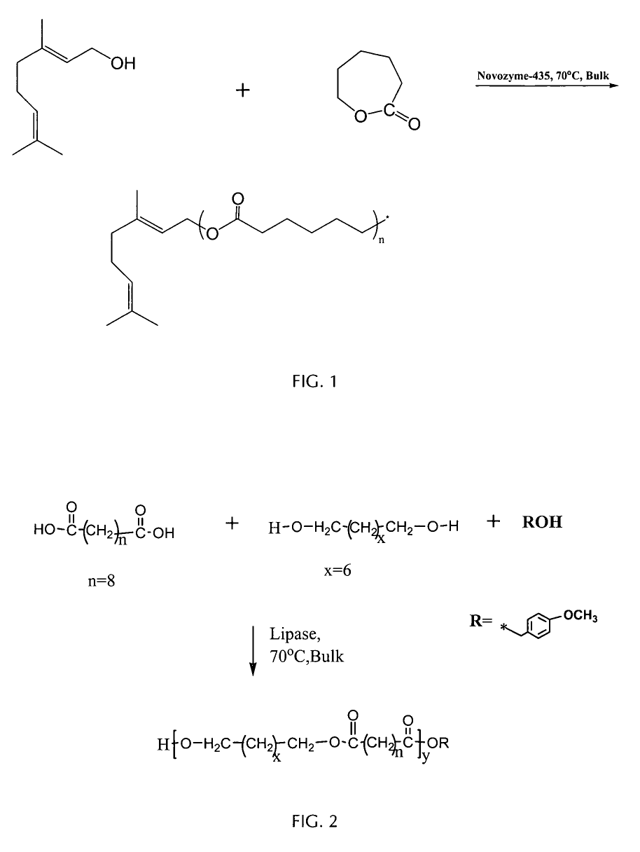

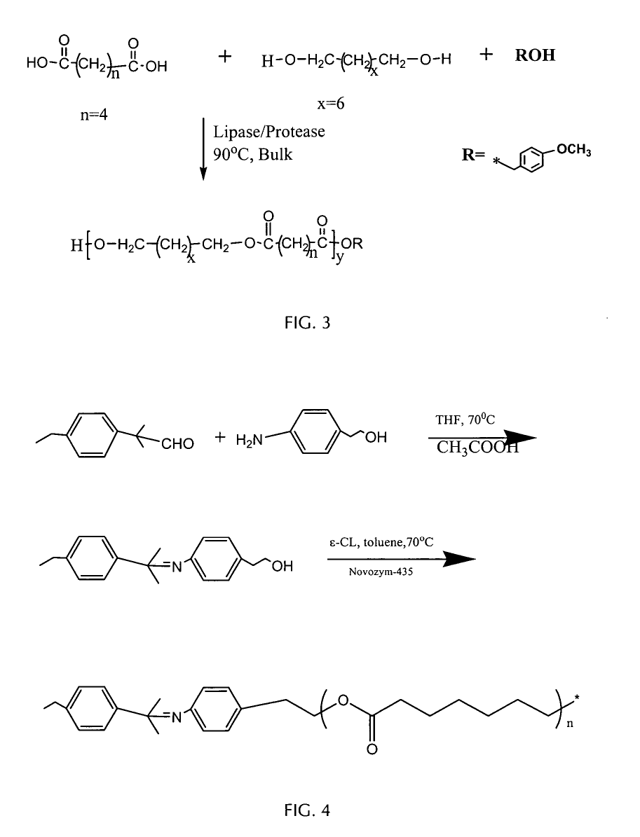

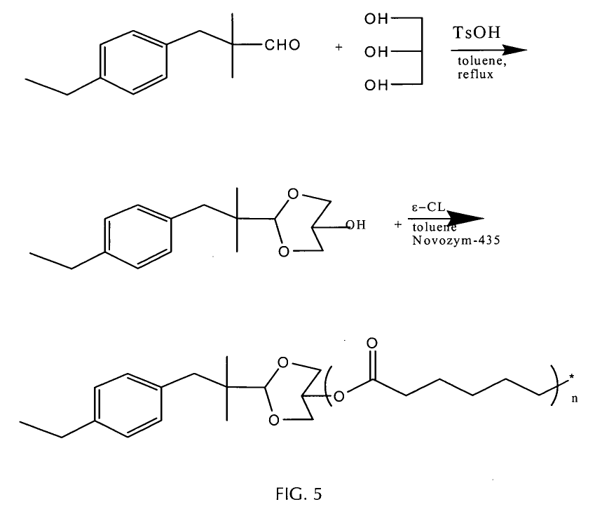

### DEFINITIONS

In this specification, various terms are defined as follows:

“Regioselective reactions” are reactions in which at least two constitutional isomers can be formed from single reactant but one isomer is observed to predominate the product of the reaction. Regioselective reactions also can include reactions in which one isomer is formed exclusively. In this invention it refers directly to the selective polymerization of two hydroxyl groups contained within a polyol that has ≧3 hydroxyl groups.

“Chemical reactions” can include the formation or dissociation of ionic, covalent, or noncovalent structures through known means. Chemical reactions can include changes in environmental conditions such as pH, ionic strength, and temperature.

A “polymer” can be and can include homopolymers, copolymers, and combinations thereof where the average chain length is greater than or equal to 2 repeat units.

An “oligomer” can be and can include homopolymers, copolymers, and combinations thereof where the average chain length is less than or equal to 10 repeat units.

A “polyol” can be any compound in which there are more than two hydroxyl groups. Polyol compounds can include compounds such as carbohydrates.

A “polyester” can be any compound in which there is more than one ester bond.

Unless otherwise defined, all technical and scientific terms used herein have the same meaning as commonly understood by one of ordinary skill in the art to which this invention pertains. Although methods and materials similar or equivalent to those described herein can be used in the practice and testing of the present invention, suitable methods and materials are described below. In addition, the materials, methods, and examples are illustrative only and not intended to be limiting.

### DETAILED DESCRIPTION OF THE INVENTION

Mammalian skin is a living organ that is capable of performing a large number of different functions, and can also be extremely reactive to materials placed in contact with it. The present invention exploits these properties of skin to achieve the prolonged release of active ingredients, both cosmetic and pharmaceutical, to the skin cells.

In the most generic approach to the concept, the delivery system of the invention comprises a topically acceptable polymer bound to a biologically active ligand by a bond. The term “polymer” as used herein encompasses homopolymers, copolymers, and combinations thereof where the average chain length is greater than or equal to 11 repeat units. The term “polymer” will also be understood to encompass oligomers, i.e., short chain polymers having a chain length of from 3 to 10 repeat units. The bond joining the active to the polymer chain, or to a monomer within the chain, may be ionic, covalent, or noncovalent of all types and should be of a nature such that it can be broken by a chemical, biological or physical process that is routinely capable of occurring on the skin, for example, enzymatic activity, or the presence of water. The polymer of the invention is characterized by comprising at least one moiety:
U-B-A
   in which U represents a physiological acceptable unit of an oligomer or polymer, B represents a bond capable of being disrupted by one of the aforementioned processes on the skin, and A represents the biological active of interest for delivery to the skin. In certain embodiments, the polymer contains on average at least two or more U units. There is no defined lower or upper limit to the polymer chain length, but typically the polymer will contain from 2 to about 40 constituent units, more typically in the range of 4 to 30 constituent units. The average molecular weight of the polymers is typically between 0.5 and 15 kDA, preferably between 1 and 5 kDA, and more preferably 3 kDA.

The use of enzymes has proven effective in facilitating mild selective polymerization reactions of lactones, cyclic carbonates, cyclic anhydrides, diacids, diesters, diols, polyacids, polyols, amino alcohols, diamines, and hydroxyacids (see for example, US 20040019178, the contents of which are incorporated herein by reference). Although the polymers of the invention can be constructed by more typical, known chemical catalysts as well, these processes are less preferred. Enzymatic reactions can be performed at low temperatures in the absence of metals. In contrast, chemical polymerizations often involve high temperatures (>150° C.), and use highly reactive organometallic reagents or catalysts that are unsafe for human contact. In addition, the harsh reaction conditions required in the chemical methods often can change the structure of an active, and thus are inimical to the retention of biological activity of the material to be delivered. Thus, preparation by enzymatic catalysis is strongly preferred, in that it avoids the use and incorporation into final products of toxic metal catalysts, it can create bonds between a biological active and the monomer/polymer that are inherently degradable in the presence of water and/or skin enzymes, and it utilizes mild reaction conditions that leave the ingredient to be delivered intact so it is fully active when released onto the skin.

The polymeric molecules contain at least one biologically active component (element A). The element A in the final product may be on the polymer's chain end(s), may be incorporated within the polymeric skeleton, and/or may be a side chains or portion thereof on the polymeric skeleton. The polymer may contain multiple active units of different chemical identities, for example, one active being positioned at the chain end, and another being positioned as a side chain. The position of any given active will depend on the identity of the active, and the identity of the U in the polymer. In other words, the final placement of the actives in the polymer depends upon the nature of the reactive group(s) available on the U units to react with the reactive group(s) available on the active.

The component U of the polymer of the invention may be any physiologically acceptable unit capable of forming part of an oligomer or a polymer and which is capable of forming a skin-disruptable bond with an active. The units most useful in the polymer are those that can be linked to the active by a covalent bond at either one or both chain ends, as a pendant group, as a repeat unit along the chain or at sites along branches of the chain. In a preferred embodiment, the units are chosen from the group consisting of lactones, cyclic carbonates, cyclic anhydrides, fatty acids, epoxides, cyclic N-carboxyanhydrides, diacids, diesters, hydroxyacids, diols, polyacids, polyols, amino alcohols, diamines, or combinations thereof. The polymers may be composed of mixtures of these units that are arranged as block copolymers, random copolymers, alternating copolymers, and any combination of these arrangements of units along chains. The polymers may have a shape or architecture that is linear, branched, brush (also referred to as comb), dendrimer or hyperbranched. For convenience, the polymeric precursors, and units thereof, will be referred to as monomers in the following text.

The component B, the bond (“skin disruptable bond”) formed between a monomer unit and an active moiety, is one which is capable of being disrupted in or on the skin by a naturally occurring physical, chemical or biological process. Such processes include, but are not limited to, enzymatic action routinely occurring on the skin, whether generated by skin cells or by cutaneous microorganisms, or hydrolysis by way of water normally present on the skin. Naturally occurring enzymes on the skin include, for example, lipases, proteases, cutinases, and esterases Examples of bonds that are readily disruptable on the skin by the natural actions of enzymes or water include, but are not limited to, ester, ether, anhydride, carbonate, amide, acetal, ketal and bonds via Schiff bases (the non-enzymatic reaction product of an aldehyde or a ketone with a primary amine). Examples of monomers that are capable of forming these bonds are lactones (e.g. E-caprolactone, para-dioxanone), epoxides (e.g. ethylene oxide), cyclic anhydrides (e.g. succinic anhydride), cyclic carbonates (e.g. trimethylene carbonate), N-carboxyanhydrides (e.g. those formed from amino acids), aldehydes (e.g. butyraldehyde), polyols (e.g. pentaerytheritol), aminoalcohols (e.g. 1-amino-4-butanol),. A particularly useful bond, with broad applicability to a variety of different active components, is an ester, and the preferred polymers of the invention are polyesters.

Component A may be selected from biologically active materials that have skin and/or general cosmetic or pharmaceutical benefits, and which are chemically amenable to the catalytic process necessary to create the disruptable bond. The active molecules, in the broadest sense, may be any which have free hydroxy, C═O, or amino groups, that will bond, under the chosen condensation reaction, with the applicable monomer unit having a free acid or free amine function. In a more particular embodiment, the active component may chemically be an alcohol, an aldehyde, a ketone or amine, or at least possess such moieties capable of binding to the monomer of interest. It will be understood that as used throughout the specification and claims herein, the term “active” shall be interpreted to include not only those materials having a direct biological activity, such as an antioxidant, or a chemical exfoliator, but also those compounds having an indirect or adjunct biological activity, such as fragrances or emollients, or any cosmetic component that has a beneficial effect when applied to the skin, whether biological or physical. Such compounds are routinely used for strictly aesthetic benefits, but may, for example, in the case of emollients, also have a physical, rather than strictly biological, benefit to the skin, or in the case of fragrances, may also have less quantifiable benefits such as mood modulation conferred by aromatherapy. Throughout the specification, the terms “active”, “bioactive” or “biologically active” are used interchangeably.

The polymers of the invention may be built directly from monomers or prepared from preformed polymers by transesterification or transamidation reactions. Such reactions can be performed in-bulk or in-solvent. The enzymes used as catalysts can be selected from those that normally function as lipases, esterases, cutinases, and proteases. Lipases, proteases and esterases are preferred. The preparation of the polymers is not limited to any one type of enzyme, and many suitable enzymes are commercially available. Useful lipases include Novozyme-435 (physically immobilized Candida antarctica Lipase B), Candida cylindreacea lipase (CCL), Candida rugosa lipase(CR), Penicillium roqueforti lipase(PR), Lipase IM (Mucor meihei), PS-30 (Pseudomonas), PA (Pseudomonas aeruginosa), Lipase PF (Pseudomonas fluorescence), immobilized lipase PC from Pseudomonas cepacia, Candida cylinderaceae lipase, porcine pancreatic lipase(PPL), and Aspergillus niger lipase. Useful proteases include α-Chymotrypsin Type II from bovine pancreas, papain, pepsin from porcine stomach mucosa, Protease Type XIII from Aspergillus saitoi, Protease (Pronase E) Type XIV from Streptomyces griseus, Protease Type VIII (Subtilisin Carlsberg) from Bacillus licheniformis, Protease Type X (Thermolysin) from Bacillus thermoproteolyticus rokko, and Protease Type XXVII (Nagarse). Lipases are particularly preferred enzymes for preparing the polymers of the invention. A particularly preferred lipase is Lipase B from Candida antarctica.

In cases where the bioactive has low volatility and high chemical stability then a chemical catalyst may be used in place of the biocatalyst. Examples of chemical catalysts that can be used for oligomerizations and polymerizations of lactones and condensation of diacid/diol systems include dimethoxydibutyltin, stannous octanoate, titanium tetrabutoxide, trialkylaluminum, monochlorodialkylaluminum, lanthanide and scandium based organometallics, and anionic systems such potassium tertbutoxide. This methodology is less preferred, however, because of the high temperatures at which they function, difficulty in purifying the products formed, the need for complete exclusion of moisture and oxygen from the polymerization reactions, a relative lack of control during polymerizations, and the formation of branched or crosslinked products when using multifunctional monomers such as sorbitol.

In one embodiment, which is elaborated more fully below, the method for preparing polymers of the invention comprises the general steps of selecting one or more monomers from the set of lactones, cyclic carbonates, cyclic anhydrides, diacids, diesters, diols, polyacids, polyols, amino alcohols, epoxides, carbohydrates, diamines, polyamines, diesters, and hydroxyacids, combining these reactants and an appropriate enzyme in a vessel, and conducting oligomerization, polymerization, transesterification, or transamidation reactions that link the reactant monomers. Appropriate enzymes for oligomerization, polymerization and transesterification reactions carried out with lactones, cyclic carbonates, cyclic anhydrides, diacids, diesters, diols, polyacids, polyols, amino alcohols, and hydroxyacids are lipases, esterases or cutinases. Appropriate enzymes for oligomerization and polymerization reactions carried out with epoxides or carbohydrates to form ether links may be performed by using glycosidases or epoxide hydrolases. Appropriate enzymes for oligomerization and polymerization reactions carried out with amino alcohols, diamines, diesters, polyacids, and polyamines include lipases and proteases. In one embodiment, the monomer mix does not contain an active component, and the active is later attached, to a chain end or a side chain, by an enzymatic or chemical reaction, as appropriate to the nature of the active. In an alternate embodiment, the active is incorporated into the monomer mix, taking part in the polymerization reaction, and being directly incorporated at one or more chain ends or as pendant groups of the polymers. In many cases, the active, unlike the other monomers, will contain only one reactive site; should this be the case, the active will then either act as an initiator for the polymerization (e.g. active with one hydroxyl will initiate lipase-catalyzed lactone polymerizations and, therefore, be located at the carboxyl terminus of the chain) or a terminator of chain extension (e.g. active with one carboxyl group that will terminate lipase-catalyzed lactone polymerizations and, therefore, be located at the hydroxyl terminus of the chain).

The reaction may be performed without the addition of solvent to the reaction vessel, if one or more of the reactants is a liquid. The enzyme, where used, is preferably an immobilized lipase maintained at approximately 70° C. The reaction may be allowed to proceed for between 1 minute and 48 hours, depending on the product desired. Preferably, between about 0.0001% to about 20% by weight of the reaction mixture consists of the immobilized catalyst, and more preferably approximately 10% immobilized catalyst where between about 5% to about 20% by weight of the immobilized catalyst is the enzyme, and more preferably approximately 0.5% catalyst where aboutl 10% by weight of the catalyst contains the enzyme.

If a solvent is used, the preferred solvents include toluene, diisopropylether and isooctane. The range of solvent used is from 0.0% to 90% by weight of the reaction mixture. Although a solvent is not necessary, using an amount of solvent approximately twice the volume of the monomer has been found to provide satisfactory results.

As an illustration of the process of the invention, copolyesters of caprolactone (CL) and polydecalactone (PDL) are prepared. The comonomers CL and PDL are transferred simultaneously into reaction vials that contain the immobilized lipase (Novozym-435), bioactive, and toluene at 70° C. The reactants are stirred and the reaction is allowed to continue for times that vary between 1 minute and 48 hours. If the reaction involves condensation between alcohol and acid groups a vacuum may need to be applied to form the product.

In addition to polymers containing a single type of linkage, e.g., a polyester, the present invention includes lipase-catalyzed synthesis of copolymers having mixed linkages such as ester/ether, ester/carbonate and ether/carbonate, which can represent the linkage between monomers, or the linkage between monomer and active, where the active is either at the chain end(s), is a repeat unit or is linked to the polymer as a pendant group. Lipase-catalyzed oligomerization or polymerization reactions may be used to form copolymers that are random, diblock, multiblock, brush, hyperbranched, dendrimers or some other arrangement of repeat units along a copolymer chain. For example, lipases may be used to catalyze polymerization reactions between combinations of structurally different moieties: i) lactones, ii) lactones with cyclic carbonates, iii) lactones with cyclic anhydrides, iv) diacids with diols, v) diacids with polyols, vi) diacids, diols and polyols, vii) diacids, diols and poly(acids), viii) chain segments that contain amino, carboxyl or hydroxyl terminal groups with with any of the above combinations of monomers. The active may be an initiator that forms the terminal groups on chains. Alternatively, the active may be a repeat unit within chains, or linked to the chain through a functional side-chain group. As noted above, the position of the active within the polymer will depend upon the nature and number of the active's reactive sites, and the nature and number of the reactive sites on the other component monomers, as well as whether one chooses to incorporate the active into the monomer mix or to add it to a preformed polymer.

Reaction parameters such as the substrates, temperature, time, solvent (or the lack of one), identity of the catalyst, preferably an enzyme, and method of catalytic activation can all be used to engineer the desired molecular weight and polymer composition. For example, provision in the reaction mix of monomer to a bioactive component with a single reaction site so that the ratio of the components is less than 5 to 1 will shorten the time of reaction, and thus potentially shorten the average chain length; alternately, the control of the ratio of the different component monomers will determine the ultimate character of the final polymer. As an example, polymerizations are performed by lipase-catalyzed ring-opening and step-condensation reactions. A preferred monomer for ring-opening polymerizations is ε-caprolactone (ε-CL). Preferred monomer pairs for ring-opening polymerizations include ε-CL/trimethylene carbonate and ε-CL/ω-pentadecalactone. Preferred diacids for step-condensation polymerizations include the following: adipic, sebacic, and dodecanoic acids; it is also possible to use in place of the acids their corresponding esters. Examples of suitable esters include methyl and ethyl esters. Many other esters of diacids can also be used that, for example, are electron withdrawing and accelerate oligomerization and polymerization reactions. Preferred diols for step-condensation polymerizations include 1,3-propanediol, 1,4-butanediol, 1,6-hexanediol, 1,8-octanediol, 1,10-decanediol, and 1,12-dodecanediol. Preferred polyols for step-condensation polymerizations include glycerol, sorbitol, and trimethylolpropane. The preferred catalyst is Novozym-435 and the preferred solvent is toluene. All of the above reactions result in the formation of copolymers that differ substantially in their solubility. For example, using hydrophobic monomers such as ε-CL and ω-pentadecalactone will permit creation of a molecule of choice that is more oil-soluble (more hydrophobic). Alternatively, use of hydrophilic monomers such as sorbitol and succinic acid will result in more water soluble (more hydrophilic) molecules. Control of rate of release of the active on skin can also be engineered by choosing, in the design of the molecule, a bond that is likely to be more quickly or more slowly degraded by an enzyme or water on the skin. For example, the use of para-dioxanone instead of ω-pentadecalactone as the monomer will result in conjugates between the bioactive and an oligomer or polymer that will more rapidly degrade by hydrolysis to release the bioactive.

The polymers of the invention may link, as its A component, any biologically active material, as generally defined above, that has an alcohol, aldehyde (preferably protected as an acetal or Schiff base), amine or carboxylic acid function (i.e. molecules having free hydroxy-, amino- or carboxylic acid groups) to an oligomer or polymer by modification of its free carboxylic acid, amine, or ester side chains (e.g. polyacrylic acid, polyvinylamine, poly[methyl acrylate] or a copolymer containing these monomers), to the chain ends of polyesters, poly(ester/carbonates), poly(ester/anhydrides) and other bioresorbable polymers. Alternatively, the A component may be incorporated as a repeat unit within chains. In broad terms, the groups defined as useful as active components may encompass exfoliating agents, vitamins, biologically active peptides, retinoids, antioxidants, anti-inflammatory agents, melanin precursors, hydroxyacids, neuromediators, antimicrobials, preservatives, fragrances, enzyme activators or inhibitors (to the extent compatible with the enzyme catalyst of the reaction or an enzyme needed on the skin for release of the active), More specific examples of such compounds include, but are not limited to alcohols, for example, vitamins such as as retinol, all-trans retinol; 3,4 didehydroretinol; calciferol and other forms of vitamin D2 and D3; whiteners such as resorcinol or resorcinol derivatives; antioxidants such as resveratrol and diols, such as sorbitol; aldehydes such as the vitamin retinaldehyde; amines, such as vitamin K or Vitamin B12, or amino acids, catecholamines, or dopamine; and acids, such as exfoliating alpha and beta-hydroxy acids (for example, lactic, glycolic, salicylic, 3-hydroxybutyric acid, 3-hydroxypropionic acid); vitamins such as nicotinic acid or retinoic acid ; whiteners such as kojic or ascorbic acid; terpenoids such as ursolic acid; hair growth stimulators such as prostaglandins and prostanoic acid ; tannins, such as caffeic acid, quinic acid, ferulic acid, rosmarinic acid, shikimic acid, ellagic acid and gallic acid; and flavones, such as genistein, apigenin, and epigallocatechin. Other examples will be immediately apparent to those skilled in the art. It will also be understood that when the present specification and claims refer to application to the skin, this is intended to encompass application to all portions of the skin and intimately associated structures, for the benefit of the stratum corneum, epidermis, dermis, the hair follicle, the hair shaft, the hair bulb, and the sebaceous glands, as well as the associated microflora and fauna.

In a particularly preferred embodiment, the A component is a fragrance component. Numerous commonly used fragrance components, both natural and synthetic, are either alcohols or aldehydes. The use of the polymers of the invention to deliver fragrance will accomplish a number of beneficial effects. First, a frequent complaint of fragrance users is that their fragrance does not last long enough. With the oligomers or polymers of the invention, the fragrance will be released over a prolonged period of time, and not all at once, as is typical with traditional fragrances, so that the benefit is appreciated over a longer time frame. Also, the delayed release of fragrances has the additional benefit of being less likely to trigger an allergic response in those individuals sensitive to certain fragrance components. Thus, the incorporation of fragrance components as part of the oligomers or polymers of the invention permits the creation of a less allergenic fragrance, a great boon to the fragrance industry. In this regard, it is particularly desirable to create oligomers or polymers incorporating those fragrance components that are frequently identified as potential allergens, such as cinnamyl alcohol, amylcinnamyl alcohol, cinnamic aldehyde, hydroxycitronellal, isoeugenol, eugenol, geraniol, benzyl alcohol, alpha-amyl cinnamic aldehyde, citral, alpha-hexyl cinnamic aldehyde, citronellol, farnesol, anise alcohol, linalool, benzyl salicylate, coumarin, hydroxyisohexyl 3-cyclohexene carboxaldehyde, benzyl cinnamates, butylphenyl methylpropional, benzyl benzoate, methyl 2-octanoate, alpha-isomethyl ionone, as well as any essential oils, or plant extracts, containing one or more of these.

For use with fragrance molecules, a preferred polymer base is one that will dissolve in an oil-like media with little water or nucleophilic components, so as to avoid premature hydrolysis of the bonds between active and oligomer or polymer before application to the skin. This will result in a formulation that will have higher shelf-life stability but will be triggered to degrade releasing the active when applied for example on skin.

Preferred Oligomers or Polymers

For Cyclic Monomers—oligomers or polymers resulting from using various components of the active with γ-valerolactone, ε-caprolactone (ε-CL), ω-octanolide, ω-decanolide, ω-dodecanolide, para-dioxanone, lactide, glycolide, β-methyl-β-butyrolactone, trimethylene carbonate, and mixtures thereof. In place of any of the above lactones can be their corresponding ω-hydroxyacids. The preferred products will be of low molecular weight (Mₙ about 2000 g/mol). By using mixtures of monomers and limiting the molecular weight the resulting products will have little or no crystallinity, will be oils that dissolve in a non-polar hydrophobic delivery media. All the above monomers and mixtures thereof can be used for this purpose. The most preferred systems contain ε-caprolactone (ε-CL) either alone or with other monomers such as trimethylene carbonate, para-dioxanone or ω-dodecanolide.

Preferred Diol/Diacid Systems

Glycerol terpolymers that consist of: i) glycerol, ii) an aliphatic diol such as 1,4-butanediol, 1,6-hexanediol, 1,8-octanediol, 1,10 decanediol or 1,12-dodecanediol, iii) an aliphatic diacid such as succinic, adipic, suberic or other chain length acid, iv) and the active that has a free alcohol groups. For example, the mono-alcohol group of certain fragrance molecules will form an ester with one or more chain end acid groups of the condensation polymer. Other forms of the active that will be used to form ester links to the condensation polymers are: i) acetals synthesized by reacting a polyol and aldehyde active that has free hydroxyl groups (e.g. acetal formed by reacting glycerol and citronellal), ii) Schiff bases with one or more free hydroxyl groups that are formed by reacting the aldehyde of an active with an aminoalcohol. The ratio of glycerol to diol and diacid will be used to vary the number of acid terminal groups. It is common knowledge to those skilled in the art that by increasing the ratio of acid to hydroxyl groups in the monomer feed the number of carboxylic acid end-groups can be increased. This is especially true since glycerol copolymers can be branched and the terminal groups of branches may have carboxylic acids. The preferred products will be of low molecular weight (Mₙ about 2000 g/mol). As above, by using mixtures of monomers and limiting the molecular weight the copolymers from condensation polymerization will be in the form of oils that dissolve in non-polar hydrophobic delivery media. All of the above monomers and mixtures thereof can be used for this purpose. The preferred systems will contain glycerol and diols/diacids with six or more carbons. Most preferred will be sebacic acid, dodecanol, or glycerol terpolymers. Terpolymers formed with high contents of diacid in the monomer feed will provide a large number of terminal acid groups to link fragrances that are alcohols, acetals of aldehyde actives with one or more “free” hydroxyl groups, Schiff bases of aldehyde actives that have one or more “free” hydroxyl groups.

Control of rate of release of the active on skin can also be engineered by choosing, in the design of the molecule, a bond that is likely to be more quickly or more slowly degraded by an enzyme or water on the skin. For example, the use of para-dioxanone instead of ε-caprolactone as the monomer will result in conjugates between the bioactive and an oligomer or polymer that will more rapidly degrade by hydrolysis to release the bioactive. The structure of Schiff bases and acetals can be engineered so that they are more rapidly or slowly hydrolyzed.

Examples of fragrances which can be bound to the oligomers or polymers are: 1) citronnellol, 2) anisol, 3) geraniol, 4) citronnellal, and 5) cinnemaldehyde. For condensation polymerizations, the alcohols citronnellol, anisol, and geraniol will react with a diacid monomer, at a propagating chain end with carboxyl terminal groups, or with the carboxyl terminal groups of pre-formed linear and branched polyesters using the monomers and reactions described above. In addition, the alcohols citronnellol, anisol, and geraniol can be used as initiators for the ring-opening polymerization of cyclic monomers such as lactones and carbonates. Alternatively, actives with aldehyde groups such as citronnellal and cinnemaldehyde may be first converted to their corresponding acetals or Schiff base derivatives by reaction with a polyol or amino alcohol, respectively. The free alcohol(s) of Schiff base or acetal derivatives can be incorporated into polymers exactly as was described for citronnellol, anisol, and geraniol. Examples of polyols are suitable for this purpose include but are not limited to erythritol, xylitol, sorbitol, lactitol, mannitol and maltitol.

### EXAMPLES

A. General Process Materials and Methods

The following provides a general disclosure of the materials and methods used in the working examples.

(i) General Protocol for Enzymatic Polymerizations Where the Cosmetic Substance is Either at the Chain End(s), is a Repeat Unit or is Linked to the Polymer as a Pendant Group.

The reactions are performed in solvent or in bulk (solventless) conditions by either the direct reaction between diols and diacids, the ring-opening of cyclic monomers such as lactones, and optionally additional compounds selected from the group consisting of polyols, hydroxy acids, lactones, carbonates, anhydrides, and combinations thereof. The mixture of selected compounds is reacted in the presence of hydrolytic enzymes and one or more actives under bulk flow condition to prepare polymers with ester links. The reaction proceeds as a simultaneous polymerization and can provide a route for direct reactions between selected compounds. The active can act as a chain initiator or terminator that is located at chain ends. Alternatively, the active can be linked through covalent bonds formed at polymer side groups or branches by enzymatic catalysis. Furthermore, the active can have multiple groups that react and form repeat units along the chain

Lipase is selected as the representative family of enzymes as it is in common use and readily extrapolated to many different reactions. The lipase (0.001 to 1% wt/wt of the monomers) is dried in a vacuum desiccator (0.1 mmHg, 25° C., 24 hr) and is transferred into a 50 mL round-bottom flask containing a homogeneous melt of a mixture that contains an alcohol or aldehyde active /polyol/diol/diacid. Alternatively the mixtures contain a homogeneous liquid of the active and cyclic monomers such as ε-caprolactone. Diesters such as the corresponding methyl or ethyl esters can be used in place of diacids. The ratio of carboxylic acid to reactive hydroxy groups is adjusted so that they are equimolar (1:1). This is accomplished by considering only the primary hydroxyl groups of the polyols as reactive. However, variation of the ratio of carboxylic acid to hydroxyl groups can be used to vary branching and the availability of free carboxyl and hydroxyl groups that are available to react with the active substance. The flasks are stoppered with rubber septa. The flasks then are placed into a constant temperature oil bath (50-100° C.) that are agitated by various means such as with magnetic stirring. For condensation polymerizations the reaction mixtures are subjected to reduced pressure (from 0.1 to 100 mmHg) to control the rate of water removal from the system.

In alternative embodiments the polyesters produced by the present process may comprise or consist of repeating units from polymerization of a cyclic monomer such as a lactone; two or more lactones; copolymerizations of lactones with cyclic carbonates; a diacid and a diol; a diacid and a polyol; a diacid, a diol and a polyol; a diacid, a diol and a hydroxy acid; a diacid, a polyol and a hydroxy acid; a diacid, a diol, a polyol and a hydroxy acid; a diacid, a dimethyl ester, a diol, and a hydroxylamine; a diacid, a diol, a hydroxylamine, and an anhydride; a diacid, a diol, a polyol, a hydroxylamine, and an anhydride, or any other suitable combination of monomers, for example combinations in which the diacid is replaced by its methylester or ethyl ester derivative. For condensation polymerizations, preferred illustrative combinations include adipic acid/1,6-hexane diol/glycerol, adipic acid/1,6-hexane diol/sorbitol, adipic acid/1,4-butanediol/dimethyladipate/ethanolamine, adipic acid/1,4-butanediol/succinic anhydride/ethanolamine, dimethyladipate/1,4-butanediol, adipic acid/ethanolamine, ethanolamine/adipic acid, diethanolamine/adipic acid, ethanolamine/dimethyladipate, N-methylethanolamine/dimethyladipate, diethanolamine/dimethyl adipate, adipic acid/glycerol, adipic acid/sorbitol, adipic acid/sucrose, adipic acid/1,4-butanediol/sorbitol, adipic acid/diethylene glycol, adipic acid/diethylene glycol/glycerol, adipic acid/diethylene glycol/sorbitol, adipic acid/diethylene glycol/trimethylolpropane, diethylene glycol/adipic acid/dimethylolpropane, adipic acid/1,6-hexanediol. Other preferred illustrative combinations can use sucrose or another carbohydrate (such as, for examplary purposes only, xylitol, or lactose) in place of glycerol or sorbitol; diacids of longer chain length (such as, for example purposes only, linear α-ω(o-diacids with 8 to 32 carbons) in place of adipic acid; diols of longer chain length (such as, for example purposes only, linear α-,ω-diols with 8 to 32 carbons) in place of 1,4-butane diol; anhydrides other than succinic anhydride such as itaconic anhydride, maleic anhydride, glutaric anhydride; alcohol amines of differing chain length other than ethanolamine (such as, for example purposes only, butanolamine, or hexanolamine); and diamines such as 1,4-diaminobutane in place of alcohol amines such as 1,4-butanolamine.

The enzyme used in the present process may be used in free form or may be bound on an inert carrier, for instance a polymer such as an anion exchange resin, cation exchange resin, an acrylic resin, polypropylene resin, polyethylene resin, polyester resin, silica resin, or polyurethane resin. When the enzyme is bound on an inert carrier it can easily be removed from the reaction mixture (e.g. by filtration) without the need for complicated purification steps. Preferably the enzyme is recovered from the reaction mixture and re-used. Preferably the enzyme is present in isolated form. Enzymes bound to an inert carrier may to some extent desorb or become detached from the carrier and diffuse into the reaction mixture.

The amount of enzyme used is not critical but the enzyme should be present in a quantity ample to catalyze the polymerization. Too little enzyme can result in longer reaction times whereas too much enzyme may be unnecessary but may result in faster reaction times. With the lipase from Candida antarctica (Novo Industries AS Catalogue no SP 435) it has been found convenient to use from 0.1 to 1.5% by weight of supported enzyme based on the total weight of monomers, preferably 0.1 to 0.6% and most preferably 0.15 to 0.3% of supported catalyst. For other enzymes, one of ordinary skill in the art can determine the appropriate amount of enzyme without undue experimentation. Furthermore, one of ordinary skill in the art can determine a suitable matrix that the enzyme can be fixed to either through covalent attachment or by other physical interactions (hydrophobic-hydrophobic, ionic, and others).

This method can be carried out at temperatures ranging from 10-120° C. Preferably, the method is carried out at a temperature between 50° C. and 100° C. Most preferably, the method is carried out at temperature between 65° C. and 90° C. It should be noted that some enzymes can denature at temperatures significantly higher than 90° C. and that some enzymes may only allow the reactions to proceed relatively slowly at temperatures below 10° C.

The method can proceed at atmospheric pressure or less than atmospheric pressure. For condensation polymerizations, the rate of water removal will affect the reaction rate. It is understood by those skilled in the art that for every polymerization there will be a optimal water content in the reaction.

The reaction in the present method can be quenched by any number of means well known to a person of ordinary skill in the art. For example, the quenching of the reaction can be accomplished by removal of the enzyme by filtration. For products of sufficiently low molar mass and viscosity this can be accomplished without the addition of a solvent. In the case of polymers that have a high melt viscosity, low levels of a solvent can be added to the polymer melt to facilitate the filtration. Alternatively, to facilitate removal and re-use of the enzyme, it can be immobilized within the reactor (e.g. reactor walls, baffles, impellors).

The total reaction time is generally from 2-48 hr, preferably from 12-24 hr. The reaction can be monitored by removing and testing samples.

(i) General Analytical Techniques

(a). Nuclear Magnetic Resonance (NMR).

Proton (¹H) and carbon (¹³C) NMR spectra were recorded on a Bruker Instruments, Inc. DPX300 spectrometer at 300 and 75.13 MHz, respectively. The chemical shifts in parts per million (ppm) for ¹H- and ¹³C-NMR spectra were referenced relative to tetramethylsilane (TMS) as an internal reference at 0.00. High-resolution ¹H- and ¹³C-1 and 2-dimensional FT-NMR, Heteronuclear ¹H—¹³C correlations, experiments were performed. One and 2-D NMR spectra were used to determine the regioselectivity of the enzymatic polyesterification reactions.

Proton NMR (in CDCl₃) was one method used to determine the number average molecular weight (Mₙ) of bioactive-poly(caprolactone) conjugates. Proton NMR signals were observed at δ5.34 and 5.09 (CH═), 4.07 (O═COCH₂), 3.64 (CH₂OH), 2.32 (O═CCH₂), other methylenes) and 1.40 (CH₃ in geraniol. The chain length by ¹H NMR end-group analysis was determined from the relative intensity of signals at 4.07 and 3.64 ppm. The molar content of geraniol in products can be determined from the relative intensity of the signals at 5.09 and 4.07. To determine the ratio of the chain-end hydroxyl and carboxyl groups the products were derivatized with oxalyl chloride and the signal at 3.64 shifted to 4.21 and a new signal at 2.9 appeared. These signals are due to the methylene carbons next to the oxalyl chloride derivatized chain-end hydroxyl and carboxyl groups, respectively. The ratio of the two signals was used to determine the relative amount of hydroxyl to carboxyl chain-ends.

(b). Molecular Weight Measurements.

Molecular weights were determined by gel permeation chromatography (GPC) using a Waters HPLC system equipped with a model 510 pump, Waters model 717 autosampler, model 410 refractive index detector, and model T-50/T-60 detector of Viscotek Corporation with 500, 10³, 10⁴ and 10⁵ A° ultrastyragel columns in series. Trisec GPC software version 3 was used for calculations. Chloroform was used as the eluent at a flow rate of 1.0 milliliters per minute. Sample concentrations of 0.2 % wt/vol and injection volumes of 100 μL were used. Molecular weights were determined based on conventional calibration curve generated by narrow molecular weight polystyrene standards obtained from Aldrich chemical company. For some of the polymer products their molecular weight was analyzed by absolute light scattering methods. Light scattering studies were also used to determine hydrodynamic constants such as the radius of gyration. These studies were performed by using ultraviolet-visible photometer, interferometric refractometer (a Wyatt OptiLab DSP), and multi-angle laser light scattering photometer (a Wyatt Dawn DSP light Scattering Instrument).

(c). Materials

(i). Diacids.

Scheme 1: HOOC—R—COOH

Where:
R₁=(CH₂)ₙCHₓ(R₁)(R₂)(CH₂)ₘ, in which
   R₁=hydrogen, keto, nitrile, halogen, thiol, disubstituted amines, trisubstituted amines, tetrasubstituted amines, carboxylic acid, hydroxyl group, acetal, ether, alkene, alkyne, isonitrile, nitrates, sulfates, phosphates, phosphoesters, and general members of the silicone family, and where R₁ may be along the chain, a pendant group that is attached directly to carbon that is along the chain, attached indirectly to the main chain through a spacer group;
   R₂=hydrogen, keto, nitrile, halogen, thiol, disubstituted amines, trisubstituted amines, tetrasubstituted amines, carboxylic acid, hydroxyl group, acetal, ether, alkene, alkyne, isonitrile, nitrates, sulfates, phosphates, phosphoesters, and general members of the silicone family;
   n=0-32, m=0-32, x=0-2;
R═CH═CH, CH₂CH═CHCH₂; and
R═(CH₂)ₓ(—Si[R′]₂—O—)ₙ(CH₂)ₓ in which
   x=1-10, n=1 to 1000, R′=methyl, phenyl, ethyl, propyl, butyl or any mixture of these groups.

Aliphatic dicarboxylic acids relevant to the present invention include R═(CH₂)ₙ where n=0 to 30. The R₁-groups may be side or pendant groups or along the main chain. R₁-groups include carbon double or triple bonds, ketones, esters, nitriles, isonitriles, nitrates, sulfates, phosphates, phosphoesters, halogens, thiols, disubstituted amines, trisubstituted amines, tetrasubstituted amines, carboxylic acid, hydroxyl group, acetal, ether, members of the family of silicone compounds (e.g. {—Si[R]₂—O—}ₙ). Examples of diacids used in this invention include, but are not limited to, oxalic acid, succinic acid, glutaric acid, adipic acid, azealic acid, sebacic acid, fumaric acid, maleic acid. In the most preferred case adipic acid is used.

(ii). Anhydrides and Hydroxyacids.

Suitable aliphatic anhydrides include but are not limited to succinic anhydride, maleic anhydride, itaconic anhydride, and pththalic anhydride. Suitable hydroxy acids include those containing from two to twenty two carbons. Preferably they contain w-hydroxyl groups but they may also contain secondary hydroxyl groups. Suitable aliphatic hydroxyl acids include but are not limited to glycolic acid, lactic acid, 4-hydroxybutyric acid, 6-hydroxycaproic acid, 8-hydroxyoctanoic acid, 10-hydroxydecanoic acid, 12-hydroxydodecanoic acid, 16-hydroxyhexadecanoic acid, 12-hydroxy stearic acids, 12-hydroxy oleic acid, 17-hydroxyloleic acid, and cholic acid. Other suitable hydroxyl acid building blocks include those commonly described as ABₓ (x=2-7) Where A and B are carboxyl and hydroxyl groups, respectively. Alternatively, ABₓ building blocks also include those where A and B are hydroxyl and carboxyl groups, respectively. Suitable AB₂ building blocks include but are not limited citric acid, maleic acid, bis-2,2 hydroxy methylpropanoic acid, malonic acid, and most preferably maleic acid.

(iii). Diols.

Scheme 2: HOH₂C—R—CH₂OH

Where:
R═(CH₂)ₙCHₓ(R₁)(R₂)(CH₂)ₘ, in which
   R₁=hydrogen, keto, nitrile, halogen, thiol, disubstituted amines, trisubstituted amines, tetrasubstituted amines, carboxylic acid, hydroxyl group, acetal, ether, alkene, alkyne, isonitrile, nitrates, sulfates, phosphates, phosphoesters, and general members of the silicone family, and where R₁ may be along the chain, a pendant group that is attached directly to carbon that is along the chain, attached indirectly to the main chain through a spacer group;
   R₂=hydrogen, keto, nitrile, halogen, thiol, disubstituted amines, trisubstituted amines, tetrasubstituted amines, carboxylic acid, hydroxyl group, acetal, ether, alkene, alkyne, isonitrile, nitrates, sulfates, phosphates, phosphoesters, and general members of the silicone family;
   n=0-32, m=0-32, x=0-2;
R═CH═CH, CH₂CH═CHCH₂;
R═C≡C, CH₂CH≡CHCH₂; and
R═HO(CH₂)ₓ(—Si[R′]₂—O—)ₙ(CH₂)ₓOH
   x=1-10, n=1 to 1000
   R′=methyl, phenyl, ethyl, propyl, butyl or any mixture of these groups.

Suitable diols for the present invention include but are not limited to α,ω-diols that contain from C-2 to C-22 carbon atoms (see Scheme 2). Diols may also include as side groups or along the chain carbon-carbon double or triple bonds, ketones, esters, nitriles, isonitriles, nitrates, sulfates, phosphoesters, halogens, thiols, disubstituted amines, trisubstituted amines, tetrasubstituted amines, carboxylic acid, acetal, ether, and members of the family of silicone compounds (e.g. {—Si[R]₂—O—}ₙ). Examples of suitable diols are ethylene glycol, poly(ethylene glycol) (e.g. molecular weight 200 Da, 1,3-propane diol, 1,4-butanediol, 1,5-pentanediol, 1,6-hexanediol, 1,8-octanediol, and 1,12-dodacanediol. The most preferable examples in these inventions are 1,4-butanediol, 1,6-hexanediol, and 1,8-octanediol.

(iv). Polyols.

The polyols in the present invention will have at least three hydroxyl groups of which at least two must be primary or highly reactive secondary hydroxyl groups. Suitable polyols includes glycerol, erythritol, pentaerythritol, xylitol, ribitol, sorbitol, 1,2,6 hexane triol, 1,2,4-butanetriol, maltose, sucrose, and lactose, with sorbitol being particularly useful. With the exception of 1,2,6 hexane triol and 1,2,4-butanetriol the polyols in the previous sentence fall within the large family of carbohydrates.

Numerous polyol monomers in pure form or as mixtures with other polyols can be used with the present method. Such monomers, as used herein, can begenerally represented by the formula Rₚ(OH)ₙ where Rₚ is the backbone of the polyol monomer and n is the number of hydroxyl groups on the polyol monomer. Preferably, Rₚ is selected so that polyol monomers have at least two lipase active hydroxyl groups that are primary or secondary hydroxyl groups, and either secondary or tertiary hydroxyl groups that are not reactive or react very slowly relative to the lipase active hydroxyl groups. Preferably the lipase active hydroxyl groups will react at least five times more rapidly than the non-active or slowly reactive secondary/tertiary hydroxyl groups. More preferably, the lipase active hydroxyl groups will react at least ten times more rapidly than the non-active or slowly reactive secondary/tertiary hydroxyl groups.

The Rₚ-group is flexible and can be selected from an array of structures. The Rₚ-group can be a carbon-based structure with between 1 to 10 carbons. The Rₚ-group can be selected from the group comprising alkanes, alkenes, alkynes. The Rₚ-group can also have multiple hydroxyl groups, be cyclic, branched, and non-branched. Furthermore, the Rₚ-group can have ketones, esters, nitriles, isonitriles, nitrates, sulfates, phosphoesters, halogens, thiols, disubstituted amines, trisubstituted amines, tetrasubstituted amines, carboxylic acids, acetals, ethers, and members of the family of silicone compounds (e.g. {—Si[R]₂—O—}ₙ). It is understood that the Rₚ-group can be substituted or unsubstituted.

Many carbohydrates are polyols that are useful in this invention as building blocks for the synthesis of polyesters with bioactives at chain terminal or branched positions. In addition, polyols can react with aldehyde bioactives to form acetals with free hydroxyl groups (e.g. reaction of an aldehyde bioactive with glycerol or mannitol). The free hydroxyl(s) of the acetal that remain after acetal formation can be used to react during oligomerizations or polymerizations that occur by either condensation or ring-opening reactions as described above. The use of polyols from natural sources is of particular interest since they are known to be safe. Exemplary sugar based polyols that are suitable for use with the present method include mannitol, glycerol, monosaccharides (e.g. glucose), disaccharides (e.g. lactose, sucrose, maltose), trisaccharides (e.g. maltotriose), poly(n-alkylglucosides) and other carbohydrate oligomers. The preferred natural polyol is glycerol.

(v). Lactones.

The lactones in the present invention include those with 4 to 16 membered rings. Suitable lactones include β- or δ-butyrolactone, γ-valerolactone, ε-caprolactone, 8-octanolide, ω-dodecanolide, ω-pentadecalactone, lactide, dioxanone and glycolide. The preferred lactone is caprolactone.

(vi). Cyclic Carbonates.

The cyclic carbonates in the present invention include trimethylene carbonate, 1-methyltrimethylene carbonate, 1,3-dimethyltrimethylenecarbonate, 2,2-dimethyltrimethylenecarbonate, 2-methyl-2-carboxytrimethylenecarbonate, 2-carboxytrimethylenecarbonate, 1,2-O-isopropylidene-[D]-xylofuranose-3,5-cyclic carbonate, 1,2-isopropylidene glucofuranose -4,4-bis-hydroxymethyl cyclic carbonate. A preferred cyclic carbonate is trimethylene carbonate.

(vii). Enzymes.

Lipases, proteases and esterases are the preferred enzyme families that can be used in this invention as catalysts for the regioselective polycondensation of sugars/diols/diacids in-bulk without activation of the acid groups. Many enzymes are commercially available and are suitable choices for use in the polymerizations described herein. They include Novozyme-435 (physically immobilized Candida antarctica Lipase B), Lipase IM (Mucor meihei), PS-30 (Pseudomonas cepacia), PA (Pseudomonas aeruginosa, Lipase PF (Pseudomonas fluorescence), lipase from Candida cylinderacea, porcine pancreatic lipase and the lipase from Aspergillus niger. Proteases such as α-Chymotrypsin Type II from bovine pancreas, papain, pepsin from porcine stomach mucosa, Protease Type XIII from Aspergillus saitoi, Protease (Pronase E) Type XIV from Streptomyces griseus, Protease Type VIII (Subtilisin Carlsberg) from Bacillus lichenifomis, Protease Type X (Thermolysin) from Bacillus thermoproteolyticus rokko, and Protease Type XXVII (Nagarse).

Other lipases and improved forms of the above lipases that may be used in this invention can be obtained by commonly used recombinant genetic methods such as error-prone PCR and gene-shuffling. Furthermore, other suitable lipases may be obtained by the mining of DNA from various environments such as in soil. The preferred enzyme in the present invention is an immobilized form of the Lipase B from Candida antarctica. Lipase B from Candida antarctica also can be used by addition to the reaction mixture in non-immobilized form. An example of a commercially available immobilized form of Lipase B from Candida Antarctica is Novozyme-435 (available from Novozymes). Other macroporous resins that may be used for the immobilization of Lipase B from Candida antarctica include silica with various modifications, Accurrel (Akzo Nobel), purolite, QDE, Amberlite. Immobilization may involve formation of a covalent bond between the enzyme and the matrix. Alternatively, immobilization may involve physical adsorption of the enzyme to the matrix by interactions such as hydrophobic-hydrophobic, ionic, or others.

### B. Examples

### Example 1

Lipase-catalyzed synthesis of oligo(caprolactone) with geraniol esterified at the carboxyl termini of chains: Novozyme-435 ( 1/10 wt/wt of monomers) dried in a vacuum dessicator (0.1 mmHg, 25° C., 24 h) is transferred under nitrogen atmosphere into oven dried 10 mL pyrex culture tubes containing ε-caprolactone and geraniol in the ratio of 5:1 mol/mol. The vials are stoppered with rubber septa and further sealed with teflon tape. Dry toluene (2:1 vol/wt of the monomers) is subsequently added into the reaction vial. The vial is then placed into a constant temperature (70° C.) oil bath with stirring for 2-4 hours. The reaction is terminated by adding excess cold chloroform and removing the enzyme by filtration (glass-fritted filter, medium pore porosity). The insoluble material is washed several times with hot chloroform. The filtrates were combined, chloroform is removed by rotary evaporation, and the residue is dissolved in chloroform:ether (1:2 v/v) and precipitated 2-times by addition to n-hexane. The resulting product is dried in a vacuum oven (0.1 mmHg, 50° C., 24 h). The product is obtained in 62% yield: M, 2170, polydispersity (M_{w}/Mₙ) 1.7, and the content of geraniol was 4 mol. %. Proton NMR (in CDCl₃): signals are observed at δ 5.34 (1H,CH═), 5.09 (1H,CH═), 4.08-4.04 (2H, t, J=6.9, O═COCH₂), 3.67-3.62 (2H, t, J=1.3, CH₂OH), 2.33-2.28 (2H, t. J=14.7, O═CCH₂), 2.09-2.04 (4H. t, J=15, CH₂ in geraniol ), 1.70-1.60 (6H, m, J=29.4, CH₂ in oligo(ε-caprolactone), 1.41-1.39 (9H, d, J=6.9, CH₃ in geraniol).

### Example 2

### Lipase-Catalyzed Condensation Polymerization of Sebasic Acid, 1,8-Octanediol, and Anisyl to Form the Corresponding Polyester with Anisyl Esters at the Carboxyl Termini of Chains

Sebacic acid (Aldrich, 2.02 g, 1 eq.) is suspended in the melt of octanediol (Aldrich, 1.32g, 0.9 eq.) at 135° C. The temperature of the reaction mixture was then lowered to 90-95° C. Anisyl alcohol (0.14g, 0.1 eq.) and Novozyme-435 (347 mg, 10% w/w of monomers) were charged to the flask and the reaction was continued for 2 h. The reaction is then subjected to reduced pressure (10 mmHg) to remove water from the system. For all other details, see the General Process Methods above. After 48 h the reaction mixture was fractionated by precipitation into methanol. The resulting product was obtained in 72% yield: Mₙ and M_{w}/Mₙ 608 and 6.5, respectively (by SEC). Proton NMR (in CDCl₃) of the fractionated product was used to analyze the polymer end-group structure (see above, general analytical techniques, NMR). This analysis showed that the molar content of anisyl alcohol is 4 mol % relative to oligo(ε-caprolactone). Furthermore, 27 mol % of chain end groups was the anisyl ester, 38 mol % are carboxylic chain ends and 35% are hydroxyl end groups. The average degree of polymerization is 8.8.

### Example 3

### Lipase-Catalyzed Condensation Polymerization of Adipic Acid, Sorbitol and Anisyl Alcohol to Form the Corresponding Polyester with Anisyl at Carboxyl Termini of Chains

Adipic acid (Aldrich, 1.46 g, 1 eq.) is suspended in the melt of sorbitol (Aldrich, 1.64 g, 0.9 eq.) at 130° C. The temperature of the reaction mixture is brought to 90-95° C. and then anisyl alcohol (0.14 g, 0.1 eq) and Novozyme-435 (324 mg, 10% w/w of monomers) were added to the reaction flask. The reaction was maintained at between 90 and 95° C. for 48 h. Furthermore, after the first 2 h, the reaction was placed under vacuum (from 20-50 mmHg) for the remaining 46 h. For all other details see the General Process Methods above. The reaction product obtained after 48 h was dissolved in chloroform, the solution was filtered to remove enzyme, concentrated, and then precipitated by addition into methanol. The product was obtained in 77% yield: Mₙ and M_{w}/Mₙ by size exclusion chromatography (SEC) were 140 and 2.9, respectively, and the molar content of anisyl alcohol was 12 mol % relative to adipate.

### Example 4

### Lipase-Catalyzed Condensation Copolymerization of Adipic Acid, Sorbitol, 1,6-Hexanediol, and Anisyl Alcohol to Form poly(1,6-Hexanoyladipate-Co-Sorbitoladipate) with Anisyl Esters at Carboxyl Termini of Chains

Into a 100 mL round bottom flask was added adipic acid (14.63 g, 1 eq.), 1,6-hexanediol (3.54 g, 0.3 eq.), and sorbitol (10.9 g, 0.6 eq). The reactants were heated with stirring at 115° C. to melt the mixture. The temperature of the reaction mixture was then lowered to 90° C. and anisyl alcohol (1.38 g, 0.1 eq.) and Novozyme-435 (3.04 g) were charged to the flask. After the first 2 h of the reaction, it was placed under vacuum (20 mm Hg) to remove water from the system. The polymerization was terminated after 24 h. The reaction mixture was dissolved in chloroform—methanol (3:1) and precipitated into diethyl ether. The product was obtained in 62% yield: Mₙ and M_{w}/Mₙ by size exclusion chromatography (SEC) were 329 and 4.0, respectively, and the molar content of anisyl alcohol was 10 mol % relative to adipate. ¹H-NMR (CD₃OD), δ 7.31-7.22 (2H, d, J=27, ArH), 6.92-6.89 (2H, d, J=12.2, ArH), 4.25-4.92 (3H,m,J=50, O═COCH₂+OCOCH), 4.092-3.47 (3H, m, J=120, HOCH₂+CHOH), 2.42-2.33 (2H, dd, J=24, OCCH₂), 1.7 (4H, brs, J=9, CH₂CH₂CO), 1.41 3H, s, OCH₃ in anisyl alcohol), 1.21-1.16 all other methylen protons. The content of anisyl alcohol in the product was determined from the relative intensity of signals at 6.8 vs. 4.32.

### Example 5

### Lipase-Catalyzed Condensation Copolymerization of Adipic Acid, Glycerol, 1,6-Hexanediol, and Anisyl Alcohol to Form Poly(1,6-Hexanoyladipate-Co-Glycerol) with Anisyl Esters at Carboxyl Termini of Chains

Adipic acid (Aldrich 1.46 g, 0.1 mole, 1 eq.) and hexane diol (Aldrich, 0.47 g, 0.4 eq.) were heated to 125° C. The temperature of the reaction mixture was brought to 90-95° C. and then glycerol (0.46 g, 0.5 eq.), anisyl alcohol (0.14 g, 0.1 eq.) and Novozyme-435 (371 mg) were charged to the reaction flask. The reaction was maintained at between 70-75° C. for 48 hr. After the first 2 h the reaction was placed under reduced pressure (20-50 mmHg) for the remaining 46 h. Further details of the method used are described above in the section entitled General Process Methods. The product formed after the 48 h reaction was dissolved in chloroform and precipitated into methanol/n-hexane (1:2). The precipitated product was obtained in 61% yield: Mₙ and M_{w}/Mₙ by size exclusion chromatography (SEC) were 374 and 6.9, respectively. The molar content of anisyl alcohol was 16 mol % relative to adipate. H-NMR (in CDCl₃), δ 6.89 (2H,ArH), 7.27 (2H, ArH), 4.21(4H, OCH₂+OCOCH₂), 3.79 (1H,CHOH glycerol), 3.5 (2H,CH₂OH), 2.35 (2H,OCCH₂), 1.67, 1.40 and 1.2 (16H).

### Example 6

### Lipase-catalyzed Synthesis of Oligo(ε-Caprolactone) with a Schiff Base Derivative of Floralozone Linked by an Ester to the Carboxyl Termini of Chains

A mixture of floralozone (1.9 g, 1 eq.), 2-(4-aminophenyl)ethyl alcohol (1.38 g, 1 eq) and 0.1 g of acetic acid in 6 mL of THF were refluxed 10 h. The solution was filtered after cooling to room temperature and the solvent was removed. Then, the residue was dissolved in ether and filtered through a glass-fritted filter (medium pore porosity). The filtrate was dried in a vacuum evaporator to give the corresponding Schiff base product in 94% yield. The Schiff base (3.1 g, 1 eq.) was then used to initiate the ring-opening polymerization of &-caprolactone (5.7 g, 5 eq.) in 4 mL toluene using Novozyme-435 (0.88 g, 10%-by-wt) as catalyst. The temperature of the reaction was 70° C. and duration was 4 hrs. The content of the reaction mixture after 4 h was dissolved in chloroform and precipitated in methanol. The product was obtained in 65% yield: Mₙ and M_{w}/Mₙ by size exclusion chromatography (SEC) were 1810 and 1.52, respectively. The molar content of floralozone in the product was 4 mol % relative to ε-caprolactone units. ¹H-NMR (in CDCl₃), δ 7.67 (1H,m,CH═N), 7.00 (2H,ArH), 6.62 (2H,ArH), 4.06 (2H, OCH₂), 3.61 (2H,HOCH₂), 2.61 (2H,COCH₂), all other methylene protons at 1.65 and 1.39 ppm, 1.056 (9H, CH₃, floralozone).

### Example 7

### Lipase-Catalyzed Synthesis of Oligo(ε-Caprolactone) with Floralozone Glycerol Acetal Linked by an Ester to Carboxyl Termini of Chains

A) Synthesis of Floralozone Glycerol Monoacetal.

A mixture of Floralozone (1.9 g, 1 eq), glycerol (1.2 g, 1.3 eq), and a few crystals of p-toluenesulfonic acid in toluene (40 mL) were added to a 2-neck 50-mL round bottom flask and heated 24 h at reflux under nitrogen with a Dean-Stark trap to remove water. The mixture was cooled, washed (bicarbonate solution and saturated NaCl solution), dried over sodium sulfate, and concentrated. The residual oil was warmed under high vacuum to remove unreacted floralozone. The yield was 72%: ¹H-NMR (in CDCl₃), δ 7.17-7.08 (4H,m,J=27.1 Hz, ArH), 4.73 (s,1H, CH in 1,3-dioxan), 4.19-3.32(5H,m, CH₂CHCH₂ in 1,3-dioxan), 2.72-2.58 (4H,m,J=42 Hz, CH₂ in floralozone), 2.02 (brs,1H,CHOH in glycerol),1.25-1.16 (3H,m,J=27Hz, ArCH₂CH₃in floralozone), 0.92-081 (6H,m,J=33Hz, CH₃ in floralozone).

B) Lipase-Catalyzed Synthesis of Oligo(ε-Caprolactone) with Floralozone Glycerol Acetal Linked by an Ester to Carboxyl Termini of Chains

Synthesized floralozone-glycerol acetal (2.6 g, 1 eq), ε-caprolactone (5.7 g, 5 eq ), toluene (10 mL), and Novozyme-435 (400 mg) were stirred at 70° C. for 4 h. The reaction was terminated by the addition of cold chloroform. Then, the enzyme was removed by filtration, chloroform was removed by roto-evaporation, the residue was dissolved in chloroform, precipitated in methanol and the precipitate was washed 2 times with ether to give the product in 68%-yield. Mₙ and M_{w}/Mₙ by size exclusion chromatography (SEC) were 675 and 7.7, respectively. The molar content of floralozone in the product is 15 mol % relative to oligo(ε-caprolactone). ¹H-NMR (in CDCl₃), δ 7.17-7.08 (4H,m,ArH), 4.66 (1H,s,CH in 1,3-dioxan), 4.11-3.35 (2H,m, OCH₂), 3.95-3.44 (5H,m, CH₂CHCH₂ in glycerol+2H, HOCH₂ in oligo(ε-caprolactone), 2.73-2.58 (2H, m, OCOCH₂in oligo(ε-caprolactone), 2.30-2.24 (4H,t, CH₂ in floralozone), 1.70-1.53 (4H,t, CH₂in oligo(ε-caprolactone),1.35-1.18 (6H, m, 2 CH₃ in floralozone+2H,CH₂in oligo(ε-caprolactone), 0.92-0.87 (3H,m,CH₃in floralozone).

### Example 8

### Lipase-Catalyzed Synthesis of Oligo(Caprolactone) with Retinol Esterified at the Carboxyl Termini of Chains

Novozyme-435 ( 1/10 wt/wt of monomers) dried in a vacuum dessicator (0.1 mmHg, 25° C., 24 h) is transferred under nitrogen atmosphere into oven dried 10 mL Pyrex culture tubes containing F-caprolactone and retinol in the ratio of 5:1 mol/mol. The vials are stoppered with rubber septa and are further sealed with Teflon tape. The vials are placed into a constant temperature (70° C.) oil bath with stirring for 2-4 hours. After the reaction temperature is reduced to 25° C., tetrahydrofuran is added to the reaction mixture. The suspended enzyme catalyst is removed by filtration (glass-fritted filter, medium pore porosity). Subsequently, THF is removed to give the product that comprises oligomers with retinol esterified at the carboxyl terminus of chains.

### Example 9

To demonstrate the utility of the polymers of the invention in accomplishing delayed release of the incorporated active agent, an experiment is conducted to demonstrate the prolonged availability of a number of different fragrance components. In particular the slow release of fragrances from polymers topically applied to the skin in appropriate formulations is observed.

In each case, 50 μL of the polymers identified below are applied to a 3 to 4 cm² patch of skin on the back side of a hand. A trained nose is required to sniff the topically applied polymers every five minutes and to record the kinetics of availability of the perfume as well as the intensity of the perceived perfume (the relative degree of availability and intensity represented in the Table by the number of ‘+’s').

The formulation contains the fragrance-polymer in an amount of 1 g in 20 ml of base, the base comprising Isopropanol −40%; jojoba oil-30%; and olive oil-30%

### Examples Are Reported in Table 1.

TABLE 1

| | Time after application (minutes) | | | | | | | |
|---|---|---|---|---|---|---|---|---|
| Compound | 0 | 5 | 10 | 15 | 30 | 45 | 60 | 90 |
| Anisol + sorbitolester | − | − | − | + | + | + | + | − |
| Geraniol + polycaprolactone | − | − | − | + | ++ | ++ | ++ | ++ |
| Citronnellol + polycaprolactone | | − | − | + | ++ | ++ | ++ | + |

These results confirm the delayed release of the fragrances bound within the polymers of the invention.

What is claimed is:

1. A polymer for topical delivery of biologically active ingredients, the polymer comprising at least one moiety:
U-B-A
in which U represents a physiologically acceptable unit of an oligomer or polymer, A represents a biologically active component, and B represents one or more bond(s) linking A to U, which bond is capable of being disrupted by a biological, physical or chemical process occurring in or on skin.

2. The polymer of claim 1 in which the moiety U-B-A is located at one or more chain ends of the polymer.

3. The polymer of claim 1 in which the moiety U-B-A is located at one or more sites within the polymer.

4. The polymer of claim 1 in which A is, or contains a moiety which is, an alcohol, an aldehyde, a ketone or amine.

5. The polymer of claim 4 in which A is or contains a moiety which is an alcohol or an aldehyde.

6. The polymer of claim 1 in which B is an ester, ether, anhydride, carbonate, amide, acetal, ketal or Schiff base bond.

7. The polymer of claim 5 in which B is an ester bond.

8. The polymer of claim 1 in which U is selected from the group consisting of lactones, cyclic carbonates, cyclic anhydrides, fatty acids, epoxides, cyclic N-carboxyanhydrides, diacids, diesters, hydroxyacids, diols, polyacids, polyols, amino alcohols, diamines, and combinations thereof.

9. The polymer of claim 8 in which U is selected from the group consisting of lactones, diacids, polyacids, diols, polyols, and combinations thereof.

10. The polymer of claim 1 in which U is selected from the group consisting of lactones, cyclic carbonates, cyclic anhydrides, fatty acids, epoxides, cyclic N-carboxyanhydrides, diacids, diesters, hydroxyacids, diols, polyacids, polyols, amino alcohols, diamines, and combinations thereof; B is an ester, ether, anhydride, carbonate, amide, acetal, ketal or Schiff base bond; and A is, or contains a moiety which is, an alcohol, an aldehyde, a ketone or amine.

11. A topical composition comprising the polymer of claim 1, in combination with a cosmetically or pharmaceutically acceptable carrier.

12. A topical composition containing the polymer of claim 10.

13. A method of delivering a biologically active component to the skin which comprises applying to the skin an oligomer or polymer for topical delivery of the biologically active ingredients, the oligomer or polymer comprising at least one moiety:
U-B-A
in which U represents a physiologically acceptable unit of an oligomer or polymer, A represents the biologically active component, and B represents one or more bond(s) linking A to U, which bond is capable of being disrupted by a biological, physical or chemical process occurring in or on skin.

14. The method of claim 13 in which A is, or contains a moiety which is, an alcohol, an aldehyde, a ketone or amine.

15. The method of claim 13 in which U is selected from the group consisting of lactones, diacids, polyacids, diols, polyols, and combinations thereof.

16. The method of claim 13 in which B is an ester, ether, anhydride, carbonate, amide, acetal, ketal or Schiff base bond.

17. A method of making a delayed release polymer having biological activity comprising the steps of (a) combining, in a reaction vessel, at least one catalyst capable of catalyzing formation of a bond B that is capable of being disrupted by a biological, physical or chemical process occurring in or on skin; at least one biological active, A; and at least one physiologically acceptable unit U capable of forming part of a polymer and which is capable of forming bond B with active A; and (b) maintaining the reaction vessel under conditions suitable for formation of bond B between A and U, thereby producing a polymer comprising at least one moiety U-B-A.

18. The method of claim in which the catalyst is an enzyme selected from the group consisting of lipases, esterases, cutinases, and proteases.

19. A delayed release polymer produced by the method of claim 17.
